(19) United States
(12) Patent Application Publication
(10) Pub. No.: US 2011/0135688 A1
(43) Pub. Date: Jun. 9, 2011

(54) Methods and Compositions for Increasing Titer of Recombinant Porcine Adenovirus-3 Vectors

(76) Inventor: Michael Sheppard, Surf Beach (AU)

(21) Appl. No.: 12/956,099

(22) Filed: Nov. 30, 2010

Related U.S. Application Data
(60) Provisional application No. 61/266,541, filed on Dec. 4, 2009.

(51) Int. Cl. A61K 39/235; C12N 15/63; C12N 5/10; C12N 15/861; A61K 31/7088; A61P 37/04

(52) U.S. Cl. 424/233.1; 435/320.1; 435/325; 435/455; 514/44 R

(57) ABSTRACT

This invention relates to methods of increasing titer of recombinant PAdV-3 produced in porcine host cells by increasing PAdV-3 encapsidation efficiency and compositions comprising the same. More particularly, the invention describes optimized recombinant PAdV-3 vectors in which the packaging signal has been optimized. In particular, the optimization involves increasing the number of packaging elements in the PAdV-3 viral genome.

### RELATED APPLICATIONS

The present application is filed as a non-provisional application of U.S. Provisional Patent Application No. 61/266,541, which was filed Dec. 4, 2009. The entire text of the aforementioned application is incorporated herein by reference in its entirety.

### FIELD OF THE INVENTION

This invention is directed to methods and compositions for increasing the titer of recombinantly produced PAdV-3 in porcine host cells. The compositions described herein are optimized recombinant PAdV-3 vectors in which the packaging signal has been optimized by increasing the number of packaging elements in the PAdV-3 viral genome.

### BACKGROUND OF THE INVENTION

The porcine adenovirus (PAdV) expression system is well-recognized as an attractive candidate for the expression and delivery of recombinant heterologous proteins. Indeed, PAdV-based vectors have been widely described as potential vaccine vectors in e.g., RE40,930, U.S. Pat. No. 7,569,217, U.S. Pat. No. 7,473,428, and U.S. Pat. No. 7,323,177.

Although many adenoviruses grow to high titers (for example, the HAdV-5 can grow to 10⁹-10¹¹ pfu/ml), it has been found that it is difficult to get porcine adenovirus type 3 (PAdV3) to grow well above 10⁶ pfu/ml in any common porcine cell lines such as ST or PK-15 cells. In order to increase the commercial viability of the PAdV3-based vectors, it would be desirable to increase the titer of recombinantly produced PAdV3.

### BRIEF SUMMARY OF THE INVENTION

The invention relates to methods and compositions for preparing and using a recombinant porcine adenovirus 3 (PAdV-3) vector wherein the vector comprises porcine adenovirus 3 ITR sequences, and at least one heterologous nucleotide sequence, wherein the PAdV-3 genome has a modified packaging signal that comprises at least three TTT^{G}/_{C} packaging elements between the 5′ inverted terminal repeat and the E1A early transcription region.

In particular embodiments, the PAdV-3 genome has been modified to include the insertion of two or more TTT^{G}/_{C} packaging elements within the region located between the 5′ inverted terminal repeat and the E1A early transcription region of PAdV-3.

In other embodiments, the PAdV-3 genome has been modified to replace the wild-type packaging signal of PAdV-3 located between the 5′ inverted terminal repeat and the E1A early transcription region of wild-type PAdV-3 with the LHE packaging signal domain from a non-PAdV-3 virus. Simply by way of example, useful packaging signals may be those from a non-PAdV-3 virus selected from the group consisting of human adenovirus type 5 (HAdV-5), canine adenovirus type 1 (CAdV-1), porcine adenovirus type 5 (PAdV5) and bovine adenovirus type 3, FAV-9 or artificial sequence.

As described in further detail herein, the packaging signal element has the general form: TTT^{G}/_{C}XₙCG wherein X is a nucleic acid sequence of length “n” and “n” is an integer between 5 and 15.

The vectors of the present invention may be replication competent or replication defective. In the replication competent vectors, the recombinant PAdV-3 may comprises a heterologous nucleotide sequence inserted into a nonessential region of the adenoviral genome. In specific embodiments, the vector comprises one or more deletion in one or more regions selected from the group consisting of the PAdV-3 E2 region, E3 region, E4 region, L1 region, L2 region, L3 region, L4 region, L5 region, L6 region and the region between E4 region and the right end of the genome. In more specific embodiments, the non-essential region is selected from the group consisting of the E3 region, ORF 1-2 and 4-7 of E4, the region between the end of E4 and the ITR of the porcine adenovirus genome.

In the embodiments in which the vector is replication defective, the recombinant PAdV-3 comprises a heterologous nucleotide sequence inserted into an essential region of the PadV-3. For example, the replication-defective vector comprises a defective or deleted E1 function.

The vectors of the invention can be prepared to contain a heterologous nucleotide sequence that is a gene that encodes a protein selected from the group consisting of an immunomodulator, an antigen, a pathogen, an immunogenic polypeptide, a therapeutic polypeptide, a growth hormone, and a cytokine.

Also contemplated is an improved recombinant PAdV-3 vector, comprising ITR sequences, packaging sequences, at least one heterologous nucleotide sequence, the improvement comprising an optimized PAdV-3 genome having at least 3 TTTG and/or TTTC packaging signal elements between the 5′ inverted terminal repeat and the E1A early transcription region of the PAdV-3 genome.

The invention further relates to a host cell comprising an adenovirus vector of the invention wherein the host cell is capable of being infected by porcine adenovirus 3, and has an increased titer of PAdV-3 production when cultured under conditions which permit packaging of the chimeric PAdV-3 into a viral capsid as compared to a host cell infected by PAdV-3 having a wild-type packaging signal that contains two or fewer TTT^{G}/_{C} elements. In specific embodiments, the cell is a mammalian cell. More specifically, the mammalian cell is a recombinant porcine cell

The invention further relates to a composition capable of inducing an immune response in a mammalian subject, the composition comprising a recombinant adenoviral vector as described herein and a pharmaceutically acceptable excipient.

The invention describes methods of use of the vectors of the invention for eliciting an immune response in a mammalian subject comprising administering a composition comprising such vectors to a mammalian subject. More specifically, the methods are for eliciting an immune response in pigs.

The methods of the invention further relate to increasing the titer of production of a recombinant porcine adenovirus in a host cell capable of being infected by porcine adenovirus 3 comprising introducing into the porcine host cells an optimized recombinant PAdV-3 of the invention, wherein presence of the optimized packaging signal in the PAdV-3 increases the titer of the recombinant PAdV-3 from the porcine host cell culture as compared to a recombinant PAdV-3 vector prepared from wild-type PAdV-3 that contains only one or two TTT^{G}/_{C} elements between the 5′ITR and the E1A region of the PAdv-3 genome.

In specific embodiments, the chimeric nucleotide sequence encoding for a non-PAdV-3 adenovirus packaging signaling region is the LHE packaging signal domain of human adenovirus type 5 (HAdV-5), comprising nucleotides 194 to 358. In other embodiments, the chimeric nucleotide sequence encoding for a non-PAdV-3 adenovirus packaging signaling region comprises a plurality of TTTG and/or TTTC elements. More particularly, the chimeric nucleotide sequence encoding for a non-PAdV-3 adenovirus packaging signaling region comprises no less than 3 TTTG and/or TTTC elements. In exemplary embodiments, the packaging signal region is any one of the nucleotide sequence represented by SEQ. ID. NOs. 1, 2, 3, 4, 6 or 7.

In the methods of the invention, the step of growing the porcine host cells in culture comprises culturing the porcine host cell under conditions which permit packaging of the chimeric PAdV-3 into a viral capsid.

More particularly, the presence of the optimized packaging signal produces at least a one log-fold, preferably a two log-fold increase in titer of the recombinant PAdV-3 in porcine cell lines as compared to growth of recombinant PAdV-3 prepared with a wild-type packaging signal. In specific embodiments, the titer of the PAdV-3 is increased to between 10⁶ pfu/ml to about 10¹² pfu/ml.

The methods may use any host cells that are permissive to infection and growth of porcine adenovirus type 3. In specific examples, these cells include but are not limited to swine testicle cell line (ST), a porcine kidney cell line (PK-15).

The invention also relates to pharmaceutical compositions that comprise the optimized porcine adenovirus type 3 (PAdV-3) pharmaceutically acceptable carrier.

### BRIEF DESCRIPTION OF DRAWING(S)

FIG. 1: Virus titers for recombinant PAV3 clones with a wildtype PAV3 or a synthetic packaging sequences expressing CMVp-PRRS ORF6-5/MLP-PRRS ORF7.

### DETAILED DESCRIPTION OF THE INVENTION

This invention relates to compositions and methods of increasing titer of PAdV-3 in porcine host cells. In brief, the PAdV-3 vector is optimized such that encapsidation efficiency of the PAdV3 genome is increased. The optimization comprises increasing the number of packaging elements of the PAdV-3 viral genome. Typical PAdV-3 titer is in the range of about 10⁶ pfu/ml. By increasing the number of TTT^{G}/_{C} elements to a more optimal level in the region between the 5′ ITR and the E1A region of the recombinant PAdV-3 genome, it is contemplated that there will be an increase in at least one log fold, and preferably more, of the titer of recombinantly produced PAdV-3. Preferably, the titer is increased to be between about 10⁶ pfu/ml to about 10¹³ pfu/ml. The methods of the invention preferably produce a titer of at least 10⁷ pfu/ml, or at least 10⁸ pfu/ml, or at least 10⁹ pfu/ml, or at least 10¹³ pfu/ml, or at least 10¹¹ pfu/ml, or at least 10¹² pfu/ml.

Adenoviruses have a characteristically conserved morphology with an icosahedral capsid consisting of three major proteins; hexon (II), penton base (III) and a knobbed fibre (IV), along with a number of other minor proteins; VI, VIII, IX, IIIa and IVa2. The virus genome is a linear, double-stranded DNA with a terminal protein (TP) attached covalently to the 5′ termini, which have inverted terminal repeats (ITRs). The virus DNA is intimately associated with the highly basic protein VII and a small peptide termed mu. Another protein, V, is packaged with this DNA-protein complex and appears to provide a structural link to the capsid via protein VI. The virus also contains a virus-encoded protease (Pr), which is necessary for processing of some of the structural proteins to produce mature infectious virus. During assembly, hexon proteins trimerize to form hexon capsomers, which are the major structural units of the capsid. These capsomers then come together with the other capsid proteins and scaffolding proteins to form the capsid. Empty capsids which contain no viral DNA or core proteins are produced in parallel to mature virions in infected cells.

Adenovirus DNA packaging requires the packaging sequence located at the left end of the viral genome. Cis-acting packaging sequence elements and trans-acting protein components in the left hand of the genome act in conjunction to mediate DNA packaging through encapsidation, which occurs in a polar manner from left to right, relying on a cis-acting packaging domain located between discrete nucleotide locations to the left of the early transcription region E1. This region in adenoviruses contains several functionally redundant sequence elements, which in human adenovirus 5 have been called ‘A’ repeats. It is believed that these A repeats enhance viral packaging. Further, it is postulated that the efficiency of the packaging process may be related to the number of A repeats that are present in the packaging domain of the viral genome

In the present invention it has been determined that the low viral titer seen in recombinant PAdV-3 particles is related to the low packaging efficiency of PAdV-3. It has been discovered herein that while other adenoviruses are recognized as having multiple A repeats within the packaging domain, PAdV-3 is unusual in that it has only one such repeat. It is contemplated that increasing the number of these repeats in the packaging domain of the recombinant virus will advantageously increase the titer of recombinantly produced PAdV-3 particles.

Thus, in specific embodiments, the present invention relates to increasing the number of TTT^{G}/_{C} repeats within PAdV-3. In another embodiment, the invention relates to increasing the packaging efficiency of recombinantly produced PAdV-3. In still other aspects, the invention relates to increased yield of encapsidated virions as compared to empty particles of recombinantly produced PAdV-3.

The invention contemplates inserting specific sequence elements such as the ‘A’ repeats of heterologous adenoviruses into the packaging domain of PAdV-3. In other embodiments, the invention contemplates engineering the packaging domain of the PAdV-3 genome to include multiple TTT^{G}/_{C} elements within the packaging domain. The invention provides methods of increasing the viral titer of recombinantly produced PAdV-3 by at least 1 log increase as compared to recombinant PAdV-3 titer of PAdV-3 particles that contain a wild-type packaging domain.

Specifically, the packaging domain sequence of PAdV-3 is located within 600 bp of the 5′ inverted terminal repeat (ITR). More particularly, the packaging domain is located between the 5′ ITR and the E1A region of the PAdV-3 genome.

In one embodiment, the cis-acting packaging domain of porcine adenovirus type 3 (PAdV)-3 is located between nucleotide position (nt) 212 and 531 of the genome at the left hand end of the genome which, although containing six AT/GC rich motifs, still results in low encapsidation efficiency in common porcine cell lines. In contrast to consensus bipartite, general structure (5′-TTTGN₈₋₁₅CG-3′) described for most packaging motifs of human adenovirus type 5 (HAdV-5), the packaging motifs I, II, III, and IV of PAdV-3 display a tripartite structure in which the continuous A/T nucleotides are flanked by G/C-rich sequences. In another embodiment, although the continuous A/T sequences are present in all of the packaging motifs, their significance in the packaging process appears to vary within each packaging motif.

The term “bipartite” refers to a packaging element having about two basic amino acids separated by a spacer of about five to about fifteen amino acids from a second cluster of usually more than about three basic amino acids. The term “tripartite” refers to a packaging element having about two basic amino acids two amino acids, separated by a spacer of about five to about fifteen amino acids from a third cluster of usually more than about three basic amino acids.

The term “encapsidation” refers to the process of incorporating a nucleic acid sequence (e.g., a vector, or a viral genome) into a viral particle. No distinction is made between the type of nucleic acid and the type of viral particle. Thus, in one embodiment, encapsidation refers to the process of placing a nucleic acid sequence (e.g., single-stranded RNA, double-stranded RNA, single stranded DNA and double-stranded DNA) into any type of viral particle, i.e., the “capsid”.

The term “viral particle” refers to the morphological form of a virus. In some virus types it comprises a genome surrounded by a protein capsid. Others have additional structures (e.g., envelopes, tails, etc.). The term “capsid” refers to a macromolecular assembly composed of viral protein subunits. The term “capsid” may include any type of viral shell, particle or coat, including a protein capsid, a lipid enveloped structure, a protein-nucleic acid capsid, or a combination thereof (e.g., a lipid-protein envelope surrounding a protein-nucleic acid capsid). In another embodiment, the viral protein subunits assemble into a viral capsid and capsid, respectively, having a structure with an inherent repetitive organization, wherein said structure is spherical in one embodiment, or tubular in another.

The term “adenovirus vector” is used interchangeably herein with the term “adenoviral vector”. The adenovirus vectors employed herein are recombinant vectors based on the genome of PAdV-3 that comprise a polynucleotide construct for the expression of a heterologous protein that is to be delivered by the adenoviral vector. Notably, however, the PAdV-3 genome has been modified not just for the expression of the heterologous protein but it has also specifically been modified so as to include more than three TTT^{G}/_{C} packaging elements in the regions located between the 5′ ITR and the E1A region of the PAdV-3 genome such that the titer of the recombinant PAdV-3 adenoviral vector is substantially increased as compared to the titer of such a vector in which the packaging signal has not been modified.

The polynucleotide construct that encodes a heterologous protein will preferably comprise DNA that encodes the protein to be delivered. Such DNA may be comprised of the nucleotide bases A, T, C, and G, but also may include any analogs or modified forms of such bases. Such analogs and modified bases are well known to those of skill in the art, and include but are not limited to methylated nucleotides, internucleotide modifications such as uncharged linkages and thioates, use of sugar analogs, and modified and/or alternative backbone structures, such as polyamides.

The terms “cis-acting sequence” and “cis-acting element” refer to DNA or RNA sequences whose functions require them to be on the same molecule. The terms “trans-acting sequence” and “trans-acting element” refer to DNA or RNA sequences whose function does not require them to be on the same molecule.

In one embodiment, there is a direct correlation between the binding affinity of P-complex for different ‘A’ repeats in vitro with the ability of the fragments to support DNA packaging in vivo. In another embodiment, TTTG, but not the CG, packaging consensus site is critical for P-complex interaction. In one embodiment, the P-complex binds to core replication sequences in the ITR. Due to its ability to interact with both packaging and core replication sequences, the cellular P-complex activity constitutes in another embodiment a trans-acting link between viral DNA replication and encapsidation.

As explained herein above, the titer of the PAdV-3 parent virus and recombinants currently being produced is relatively low as compared to the titer observed in for example the recombinant production of HAdV5. This could be due to a variety of reasons. In one embodiment one such reason is the apparent lack of an Adenovirus Death Protein (ADP) which is involved in the lysis of the host cell following viral replication of HAdVs. Still another explanation for the low titer of PAdV-3 vectors is that the Adenovirus DNA is packaged into the virus particle inefficiently resulting in large numbers of empty particles.

There are at least 4 components required for efficient DNA packaging in an adenovirus: (1) Inverted terminal repeats (ITR); (2) The LHE packaging signal; (3) Terminal protein (TP); and (4) the packaging protein IVa2.

In its most basic form, the consensus packaging signal is a series of TTT^{G}/_{C} sequence repeats in the 5′ end of the adenovirus genome between the inverted terminal repeat (ITR) and the start of the E1A region. The E1A region has two major components sharing substantial stretches of sequence that in one embodiment, are termed 289R (or 13S) and 243R (or 12S), based on the number of amino acid residues. These E1A proteins modulate in one embodiment, cellular metabolism to make the cell more susceptible to virus replication. In one embodiment, the packaging signal motif sequence is repeated 6 or 7 times and in other certain embodiments, there are slight variations of the TTTG sequence such as TTTC.

The term “packaging signal” refers to the non-coding, cis-acting sequence required for encapsidation of PAdV-3 DNA strands during viral particle formation. In PAdV-3, this sequence has been mapped to loci extending from upstream of the 5′ ITR to at least the tatata start codon of the E1A.

In HAdV-5 there are seven TTTG and possibly 2 others (see sequences below). Canine adenovirus (CAdV-1) and bovine adenovirus (BAdV-3) show these packaging signals to be present (see sequences below). However, they are essentially absent from PAdV-3, apart from 1 TTTG (see sequence below). It is recognized herein for the first time that the presence of 1 TTTG (and maybe 2) in PAdV3 indicates that the DNA packaging, albeit still extant, is very inefficient and thus would result in large numbers of empty capsids. The present invention recognizes and exploits this feature in increasing the titer of recombinantly produced PAdV3 by increasing the number of packaging signals that are present in the recombinant PAdV3 vector. SEQ ID No. 1 below depicts the left hand end (LHE) (5′) of human adenovirus serotype 5 (HAdV-5):

(SEQ ID NO. 1)
catcatcaat aatatacctt attttggatt gaagccaata
tgataatgag ggggtggagt ttgtgacgtg gcgcggggcg
tgggaacggg gcgggtgacg tag tagtgtg gcggaagtgt
gatgttgcaa gtgtggcgga acacatgtaa gcgacggatg
tggcaaaagt gacgtttttg gacgttTTTG gtgtgcgccg
gtgtacacag gaagtgacaa ttttcgcgcg gttttaggcg
gatgttgtag taaaTTTGgg cgtaaccgag taagaTTTGg
ccattttcgc gggaaaactg aataagagga agtgaaaTCT
GaataatTTT Gtgttactca tagcgcgtaa taTTTGtcta
gggccgcggg gacTTTGacc gTTTAcgtgg agactcgccc
aggtgttttt ctcaggtgtt ttccgcgttc cgggtcaaag
ttggcgtttt attattatag

As can be seen from the above sequence, HAdV-5 contains at least 7 TTTG packaging elements and possibly 2 others. The titer of recombinantly produced HAdV-5 is in the range of 10⁹-10¹¹ pfu/ml.

Likewise, the LHE of canine adenovirus type 1 (CAdV-1) shown below in SEQ ID NO: 2, contains at least 6 TTT^{G}/_{C} elements:

catcatcaat aatatacagg acaaagaggt gtggcctaaa
tgttgttttt ttttttaaaa agtttttgtt tgattgtttt
gacaaggtta caccctgttc agggcgtttc ccacgggaaa
gaccatgacg tcaattgggt gtttttgtgg actttggccc
gcgagcccgt tTTTGactgt ttactgaaat tgtggttTTT
Gggtggagtg tttactcatg tcagaggaaa tTTTCtgggt
ttattgttct agccttggag ccaatTTTCc acacccggcg
ccagactgtc tgacattgtT TTGagaaact ctccgttccc
agggtcacag gaccgcaccc gcgagaatgg tcacgcctTT
TGttttttct gctttaaatt cactgcagag acgaaagagg

The LHE of bovine adenovirus type 3 (BAdV-3) shown below as SEQ ID No. 3 contains ten TTT^{G}/_{C} elements:

catcatcaat aatctacagt acactgatgg cagcggtcca
actgccaatc atttttgcca cgtcatttat gacgcaacga
cggcgagcgt ggcgtgctga cgtaactgtg gggcggaggc
gtcgcgg agg cggcggcgct gggcggggct gagggcggcg
ggggcggcgc gcggggcggc gcgcggggcg gggcgagggg
cggagttccg cacccgctac gtcatTTTCa gacatttttt
agcaaaTTTG cgcctTTTGc aagcattTTT CtcacaTTTC
aggtatttag agggcggatt TTTGgtgttc gtacttccgt
gtcacatagt tcactgtcaa tcttcattac ggcttagaca
aatTTTCggc gtctTTTCcg ggtttatgtc cccggtcacc
tttatgactg tgtgaaacac acctgcccat tgtttaccct
tggtcagttt TTTCgtctcc tagggtggga acatcaagaa
caaaTTTGcc gagtaattgt

The LHE of porcine adenovirus type 5 (PAdV-5) shown below as SEQ ID No. 4 contains 7 TTT^{G}/_{C} elements.

cccatgggtg aggtctggac atgcccagac acgtcagagc
taTTTGacaa aggtcacTTT Cgaccgcgcc tgtggaaTTT
Cccccgtaca gcccatTTTC gtcatggact ccgcctaTTT
Cacactataa gagcaggcga cacagttctc tccacattcg
attcgagact cgccgtacac ggacacacca caaagtaagt
tgaaatgagt tcagacaggc ttcttTTTGt gaggcTTTGt
agggacgctg ttcctcctcg ccgggcatcg ccaggcgctg
ctggttatga tttatgttct acaaactacg tagtcgtgag
ggcgcgggat.

The LHE of PAdV-3 shown below as SEQ ID NO:5 is shown below as SEQ ID NO:5:

catcatcaat aatataccgc acacttttat tgcccctttt
gtggcgtggt gattggcgga gagggttggg ggcggcgggc
ggtgattggt ggagaggggt gtgacgtagc gtgggaacgt
gacgtcgcgt gggaaaatga cgtg tgatga cgtcccgtgg
gaacgggtca aagtccaagg ggaaggggtg gagccctggg
gcggtcctcc gcggggcggg gccgagcggc ggaaattccc
gcacaggtgg agagtaccgc gggatTTTGt gccctctgga
ccggaccttc gccctccggt gtggcacttc cgcaccacac
gtccgcggcc cggtattccc cacctgacga cggtgacacc
actcacctga gcggggtgtc cttcgcgctg agaggtccgc
ggcggccgcc cgagatgacg tgtgtgggtg tatttTTTCc
cctcagtgta tatagtccgc.

The packaging sequence used may in other examples be the signal from Fowl Adenovirus 9 (FAV9) located from upstream of the 5′ ITR to at least the tatata start codon of the E1A.

The LHE of fowl adenovirus type 9 (FAdV9) shown below as SEQ ID No. 7:

catcatctat atatacctac atgaatgacg ctgctgtcac
ctcgcactac cgctcccgcc cgctagatgg ca aaattgct
ccaccaaagt atttccgtga tacaaagtcc ggactgggcg
cggcgaatac gtgccgggcg aggcacggag ccgactttct
ggcaccgata gcaataaatg ggcgttccct tgcgtgcgga
attgcgccgg cacagtccca atggcttggg agcggtccag
aaagcgatta gtttttgcgt acgcacgcga aagctatttt
tgcagcactt acttccttat ttgtttacaa ccgcctccgc
ctgtgtgtaa tttaaaattt gtacataccg ttccgatcgc
cgtacttggg cttggaacgc ggacttcgag tccctaaatt
gatttatggt gtcttaatca gcgctttttg tgccacgttg
tgcgttctcc gttgaccttt gaaaaggcgc gtggtatttc
tcggtgtgtt tttcgggcgg attacgcgtt acgtgatcgt
tttatggcgg gctccgggcg TATAacagtg gagcggctga
tcgtcagagc acggatgagc gagcagccgg actcaccgcc
ggagctctat ccgtctctcc tggtcctgcg ggtgaacccg
gttccccgta ctccgttcgt tttggacatc agccgtttgc
ccgtggtgcc ggagcgcatt ttgctgcacg actttccgaa
cggaccctgg tcggaaacga tttggtacgg gaagatcact
cagcgggagc tgaaccaggc gctagagtct atcgtggacg
tgctttaggt aatttcctcc gttctttttc ccgtttaggt
gaggagATG

Nucleic acids 1-72 represented the ITR and there is a possible TATA at position 541 and position 847 is a recognizable ATG site. The TTTGs are shown at 241, 200, 361 (ATTG) and 421.

In the sequences shown above, the italicized sections represent the inverted terminal repeats and underlined bold sequences represent the start of the E1A transcription region. In contrast to HAdV-5, CAdV-1, BAdV-3, PAdV-5 each of which contain multiple TTT^{G}/_{C} elements in the packaging signal positioned between the ITR and the start of the E1A region of the LHE of the viral genome, PAdV-3 contains only 2 such elements. In the present invention the inventor has increased the number of TTT^{G}/_{C} elements in PAdV-3 to at least 3, 4, 5, 6, 7, 8, 9 or 10 such elements located between the LHE ITR and the start of the E1A region of PAdV-3. In this manner, it is expected that the titer of the recombinantly produced PAdV-3 is increased at least by one log fold. Thus, in preferred embodiments, the titer of recombinantly produced PAdV-3 virus from such optimized vectors is at least about 10⁷ pfu/ml, more preferably, it is at least about 10⁸ pfu/ml, more preferably, it is at least about 10⁹ pfu/ml, more preferably, it is at least about 10¹⁰ pfu/ml, more preferably, it is at least about 10¹¹ pfu/ml, more preferably, it is at least about 10¹² pfu/ml or even about 10¹³ pfu/ml.

The packaging elements act as independent functional units in viral DNA packaging. Accordingly, provided herein is a method of increasing the titer of a recombinant porcine adenovirus serotype 3 (PAdV-3) in a porcine host cell culture, the method comprising the steps of: introducing an optimized PAdV-3 into said porcine host cells, wherein the optimized PAdV-3 comprises no less than 3 TTTG and/or TTTC packaging signal sites operably linked between the 5′ inverted terminal repeat (5′ ITR) and the E1A early transcription region; expressing said optimized PAdV-3; infecting the porcine host cells expressing the optimized PAdV-3; growing said infected porcine host cells in culture; recovering the optimized PAdV-3 from the culture, thereby increasing encapsidation efficiency and increasing expression and yield of a recombinant PAdV-3 in a porcine host cell culture.

The spacing between adjacent packaging signal sites is optimized specifically for PAdV-3, by assuring a GC nucleotide tandem at a fixed distance to the 3′ side of the sequence. In another embodiment, the fixed distance is between 5 and 15 residues, yielding an extended packaging sequence site having the general form of 5′-TTT[G/C]XₙGC-3′ wherein n is between 5 and 15. Accordingly, and in one embodiment, the nucleotide sequence represented by SEQ ID No. 5 is modified by inserting up to six additional packaging sequence signal repeats, while maintaining the original location of the CG dinucleotide 12 nucleotides downstream on the 3′ side (5′-TTT[G/C]X₁₂GC-3′) yielding:

(SEQ ID No. 6)
catcatcaat aatataccgc acacttttat tgcccctttt
gtggcgtggt gattggcgga gagggttggg ggcggcgggc
ggtgattggt ggagaggggt gtgacgtagc gtgggaacgt
gacgtcgcgt gggaaaatga cgtgtgatga cgtcccgtgg
gaacgggtca aagtccaagg ggaaggggtg gagccctggg
gcggtcctcc gcggggcggg gccgagcggc ggaaattccc
gcacaggtgg agagtaccgc gggat [symbol] t gccctctgga
ccggaccttc gcccTTTGgt gtggcacttc cgcaTTTGac
gtccgcggcc cggtTTTGcc cacctgacga cggtgTTTGc
actcacctga gcggggtgtc cTTTGcgctg agaggtccgc
ggTTTCgcc cgagatgacg tgtgtgggtg tattt [symbol] c
cctcagtgTA TATAgtccgc

wherein the italicized sequence represents the ITR, and the TATA box represents the start of the E1A transcription region. Bold and underlined GC dinucleotide represent potential sites for inserting packaging signal sequences to maintain the original 12 nucleotide spacing between the 3′ end of the packaging signal sequence. In one embodiment, since the IVa2 protein activity is serotype specific, maintaining the original spacing helps the additional recognition by the packaging machinery of the inserted sites as a packaging signal.

In specific embodiments, the invention also contemplates a chimeric PAdV-3 vector, optimized for increased yield and/or expression when compared to a wild type (wt) PAdV-3, comprising a chimeric nucleotide sequence encoding for a non-PAdV-3 adenovirus packaging signaling region, comprising the region between 5′ ITR and E1A of the non-PAdV-3 adenovirus operably linked to the 5′ side of the early transcription region E1_{A} of the PAdV-3.

The methods of the invention may be used to increase expression and/or yield of a recombinant PAdV-3 in a porcine host cell culture, the method comprising the steps of: introducing an optimized PAdV-3 into said porcine host cells, wherein the optimized PAdV-3 comprises a chimeric nucleotide sequence encoding for a non-PAdV-3 adenovirus packaging signaling region, comprising the region between 5′ ITR and E1A of the non-PAdV-3 adenovirus operably linked to the 5′ side of the early transcription region E1_{A} of the PAdV-3; infecting the porcine host cells expressing the optimized PAdV-3; growing said infected porcine host cells in culture; and recovering the optimized PAdV-3 from the culture, thereby increasing encapsidation efficiency and increasing expression and yield of a recombinant PAdV-3 in a porcine host cell culture.

In one embodiment, increasing the expression and/or yield, refers to at least a 10-fold increase in the pfu/ml of mature PAdV-3 capsids in the host porcine cells. The porcine cells in which the PAdV-3 capsids are prepared may be any porcine cells that can be infected with and support the preparation of PAdV-3 virions. In exemplary embodiments, the PAdV-3 is expressed in swine testicular cells (ST). In other embodiments, the PAdV-3 is expressed in porcine kidney cells (PK-15). Typically, recombinant produced PAdV-3 that has not been optimized for packaging as described herein is produced in these cells at a titer of about 10⁶ pfu/ml, the present invention that optimizes the packaging of the PAdV-3 results in an increase in viral titer in swine testicular cells (ST) and/or porcine kidney cells (PK-15) at least a level of 10⁷ pfu/ml. The skilled artisan would recognize that an increase of one order of magnitude in the yield of capsids per unit volume, or according to the method chosen to measure viral particle yield is within the scope of optimization of yield and expression as described herein. In one embodiment, host porcine cells as used herein also encompass specific porcine tissue culture, such as lung, gut, colon, oronasal, stomach and the like.

Preparations of PAdV-3 typically will contain at least some empty capsids in addition to the filled virions containing the viral genome. The filled capsids can be readily separated and the percentage of empty capsids can be analyzed. E.g. gel electrophoresis, full capsids migrate slightly faster than the empty capsids and the percentage of empty particles and full capsids can be determined to analyze the packaging or encapsidation efficiency. In another embodiment, since full capsids have a higher density than empty capsids, measurement of encapsidation or packaging efficiency is carried out using equilibrium density-gradient sedimentation, or reverse phase HPLC in yet another embodiment.

The IVa2 protein binds to the repeat sequence motifs in the packaging sequence. The IVa2 protein and the rest of the adenovirus packaging machinery is serotype specific. Accordingly, provided herein is a chimeric PAdV-3 vector, optimized for increased titer when compared to a PAdV-3 that has a wild-type packaging domain between the 5′ITR and the E1A region, comprising a chimeric nucleotide sequence encoding for a non-PAdV-3 adenovirus packaging signaling region comprising: the 5′ ITR region; the packaging signal region; and the IVa2 region of the non-PAdV-3 adenovirus operably linked to the 5′ side of the E1A region of the PAdV-3 genome.

The term “chimeric” refers in one embodiment to any gene or DNA that contains DNA sequences, including regulatory and coding sequences that are not found together in nature. In another embodiment, the term “chimeric” refers to sequences encoding parts of proteins not naturally adjoined. In yet another embodiment, the term “chimeric” refers to parts of promoters that are not naturally adjoined. Accordingly, in one embodiment, a chimeric DNA sequence may comprise regulatory sequences and coding sequences that are derived from different sources, or comprise regulatory sequences and coding sequences derived from the same source, but are arranged in a manner different from that found in nature. In one embodiment, a chimeric DNA sequence may comprise regulatory sequences and coding sequences that are derived from the same source, but where certain encoding regions are repeated in a manner different from that found in nature.

“Naturally occurring” is used in one embodiment to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

In one embodiment, the term “vector” refers to, inter alia, any plasmid, cosmid, phage, capsid or binary vector in double or single stranded linear or circular form which may or may not be self transmissible or mobilizable and which can transfect prokaryotic or eukaryotic host either by integration into the cellular genome or exist extrachromosomally (e.g., autonomous replicating plasmid with an origin of replication). The term “viral vector” and the like refers in another embodiment, to a recombinantly produced virus or viral particle that includes a polynucleotide to be delivered into a host cell, either in vivo, ex vivo or in vitro.

In another embodiment, the term “Operably-linked” refers to the association of nucleic acid sequences on single nucleic acid fragment so that the function of one is affected by the other. In one embodiment, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter).

The terms “optimized PAdV-3”, “optimized PAdV-3 coding sequence” and the like refer to a modified PAdV-3 coding sequence, wherein the modified PAdV-3 coding sequence results in expression of PAdV-3 capsids in higher titer, or increased amounts relative to the native PAdV-3.

The packaging signal sequence used in the methods and recombinant PAdV-3 vectors described herein may be any TTT^{G}/_{C} viral sequence that can be inserted between the 5′ITR and the E1A region of the PAdV3 genome. In exemplary embodiments, the PAdV3 genome is modified to include additional TTT^{G}/_{C} elements within the native packaging signal that is located between the 5′ITR and the E1A region of the PAdV3 genome. In other embodiments, the native PAdV3 sequence that is located between the 5′ITR and the E1A region of the PAdV3 genome is replaced by the analogous sequence located between the 5′ ITR to the E1A region of human adenovirus type 5 (HAdV-5). In another embodiment, the native PAdV3 sequence that is located between the 5′ITR and the E1A region of the PAdV3 genome is replaced by the analogous sequence located between the 5′ ITR to the E1A region of canine adenovirus type 1 (CAdV-1). In still another embodiment, the native PAdV3 sequence that is located between the 5′ITR and the E1A region of the PAdV3 genome is replaced by the analogous sequence located between the 5′ ITR to the E1A region of porcine adenovirus type 5 (PAdV-5). In still another embodiment, the native PAdV3 sequence that is located between the 5′ITR and the E1A region of the PAdV3 genome is replaced by the analogous sequence located between the 5′ ITR to the E1A region of bovine adenovirus type 3 (BAdV-3).

In specific embodiments, the recombinant PAdV-3 genome is modified such that a chimeric adenovirus LHE packaging signal domain inserted at the 5′ side of the PAdV-3 E1A promoter site to optimize the expression and/or yield of the recombinant PAdV-3. Thus, in some embodiments, the entire LHE packaging signal domain of human adenovirus type 5 (HAdV-5), comprising nucleotides 194 to 358 from the 5′ terminus is used to replace the PAdV-3 LHE.

In specific embodiments, the embodiment, the chimeric adenovirus LHE packaging signal domain inserted to the 5′ side of the PAdV-3 E1A promoter site, to optimize the expression and/or yield of the recombinant PAdV-3 in the methods described herein, is the sequence represented by SEQ ID NO: 1. In other embodiments, the chimeric adenovirus LHE packaging signal domain inserted to the 5′ side of the PAdV-3 E1A promoter site, to optimize the expression and/or yield of the recombinant PAdV-3 in the methods described herein, is the sequence represented by SEQ ID NO: 2. In another embodiment, the chimeric adenovirus LHE packaging signal domain inserted to the 5′ side of the PAdV-3 E1A promoter site, to optimize the expression and/or yield of the recombinant PAdV-3 in the methods described herein, is the sequence represented by SEQ ID NO: 3. In another embodiment, the chimeric adenovirus LHE packaging signal domain inserted to the 5′ side of the PAdV-3 E1A promoter site, to optimize the expression and/or yield of the recombinant PAdV-3 in the methods described herein, is the sequence represented by SEQ ID NO: 4. In another embodiments, In another embodiment, the chimeric adenovirus LHE packaging signal domain inserted to the 5′ side of the PAdV-3 E1A promoter site, to optimize the expression and/or yield of the recombinant PAdV-3 in the methods described herein, is the sequence represented by SEQ ID NO: 6. Each of SEQ ID NO:1, 2, 3, 4, and 6 is a discrete embodiment of the LHE packaging signal domain that may replace the 5′ side of the PAdV-3 E1A promoter site, to optimize the expression and/or yield and/or encapsidation efficiency of the recombinant PAdV-3 in the methods described herein.

In one embodiment, the optimized PAdV-3 comprising a chimeric nucleotide sequence encoding for a non-PAdV-3 adenovirus packaging signaling region, comprising the region between 5′ ITR and E1A of the non-PAdV-3 adenovirus operably linked to the 5′ side of the early transcription region E1A of the PAdV-3, used in the methods of increasing the expression and/or yield and/or the encapsidation efficiency provided herein, further comprises a selected transgene under the control of regulatory sequences directing expression of said transgene.

The adenovirus vectors used herein may be replication-competent or replication-defective in a target cell. In the event that the vectors are replication-defective, the vectors may require use of a helper cell or a helper virus to facilitate replication. Use of helper cells or helper viruses to promote replication of replication-defective adenoviral vectors is routine and well-known in the art. Typically, such helper cells provide the function of the entity that has been knocked out of the recombinant adenoviral vector to render it replication defective.

A replication competent vector on the other hand may be referred to as a “helper-free virus vector” in that it does not require a second virus or a cell line to supply something defective in the vector. As noted above, the present invention is used to increase the titer of the recombinant PAdV-3 recombinant adenoviral vector such that greater than 10⁶ pfu/ml is produced so as to provide an advantage of greater production as compared to use of a PAdV-3 vector that comprises a wild-type PAdV-3 packaging signal. The transgene being expressed by the PAdV-3 is not important to the increased packaging properties provided herein and may be any transgene that is desired to be expressed by the recombinant PAdV-3. The transgene may be incorporated into a non essential site at the E3 region of PAdV-3. In one embodiment, the insertion site is at the ITR region between map units 97 and 99.5.

The preparation of the recombinant PAdV-3 vectors as vaccines or vectors for the recombinant expression of the transgene inserted therein is limited only by the insertion capacity of the PAdV-3 genome and ability of the recombinant PAdV-3 vector to express the inserted heterologous sequences. In general, adenovirus genomes can accept inserts that increase the size of the recombinant adenovirus to be approximately 105% of the wild-type genome length and remain capable of being packaged into virus particles. The insertion capacity can be increased by deletion of non-essential regions and/or deletion of essential regions, such as, for example, EI function, whose function can then be provided by a helper cell line, such as one providing EI function. In some embodiments, a heterologous polynucleotide encoding a protein is inserted into an adenovirus E3 gene region. In other embodiments, the non-essential portions of the E3 region are deleted and the heterologous polynucleotide encoding a protein is inserted at that gap left by the deletion. In some preferred embodiments, where the recombinant adenoviral vector is a PAdV-3 based adenoviral vector, the heterologous gene can be inserted into the region of the PAdV-3 genome selected from the group consisting of PAdV-3 E2 region, PAdV-3 E3 region, PAdV-3 E4 region, PAdV-3 L1 region, PAdV-3 L2 region, PAdV-3 L3 region, PAdV-3 L4 region, PAdV-3 L5 region, PAdV-3 L6 region and the region between PAdV-3 E4 region and the right end of the PAdV-3 genome. The sequences of each of these regions of PAdV-3 are well known to those of skill in the art and have been characterized in e.g., RE40,930, U.S. Pat. No. 7,569,217, U.S. Pat. No. 7,473,428, and U.S. Pat. No. 7,323,177.

Other regions into which the heterologous gene may be inserted include the E4 region. Where the recombinant adenoviral vector is a PAdV-3 based vector, the entire PAdV-3 E4 region, except that region that encodes ORF3 can be deleted to make room for the heterologous gene. As shown in Li et al. (Virus Research 104 (2004) 181-190), the PAdV-3 E4 region located at the right-hand end of the genome is transcribed in a leftward direction and has the potential to encode seven (pl-p7) ORFs. Of these only ORF p3 is essential for the replication. As such, much if not all of the rest of the E4 region may readily be deleted without rendering the virus replication defective, thereby allowing for more room for heterologous inserts. In one embodiment of the invention, insertion can be achieved by constructing a plasmid containing the region of the adenoviral genome into which insertion is desired, such as a polynucleotide encoding for a desired therapeutic protein. The plasmid is then digested with a restriction enzyme having a recognition sequence in that adenoviral portion of the plasmid, and a heterologous polynucleotide sequence is inserted at the site of restriction digestion. The plasmid, containing a portion of the adenoviral genome with an inserted heterologous sequence, is co-transformed, along with an adenoviral genome or a linearized plasmid containing the adenoviral genome into a bacterial cell (such as, for example, E. colï). Homologous recombination between the plasmids generates a recombinant adenoviral genome containing inserted heterologous sequences. In these embodiments, the adenoviral genome can be a full-length genome or can contain one or more deletions as discussed herein.

Deletion of adenoviral sequences, for example to provide a site for insertion of heterologous sequences or to provide additional capacity for insertion at a different site, can be accomplished by methods well-known to those of skill in the art. For example, for adenoviral sequences cloned in a plasmid, digestion with one or more restriction enzymes (with at least one recognition sequence in the adenoviral insert) followed by ligation will, in some cases, result in deletion of sequences between the restriction enzyme recognition sites. Alternatively, digestion at a single restriction enzyme recognition site within the adenoviral insert, followed by exonuclease treatment, followed by ligation will result in deletion of adenoviral sequences adjacent to the restriction site. A plasmid containing one or more portions of the adenoviral genome with one or more deletions, constructed as described above, can be co-transfected into a bacterial cell along with an adenoviral genome (full-length or deleted) or a plasmid containing either a full-length or a deleted genome to generate, by homologous recombination, a plasmid containing a recombinant genome with a deletion at one or more specific sites. Adenoviral virions containing the deletion can then be obtained by transfection of mammalian cells including but not limited to the stably transformed cells containing the additional fibre gene described herein, with the plasmid containing the recombinant adenoviral genome. The insertion sites may be adjacent to and transcriptionally downstream of endogenous promoters in the adenovirus. An “endogenous” promoter, enhancer, or control region is native to or derived from adenovirus. Restriction enzyme recognition sequences downstream of given promoters that can be used as insertion sites, can be easily determined by one of skill in the art from knowledge of part or all of the sequence of adenoviral genome into which the insertion is desired. Alternatively, various in vitro techniques are available to allow for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31:163-186.

Expression of a heterologous sequence (alternatively referred to herein as the “transgene”) inserted at a site that is not downstream from an endogenous promoter also can be achieved by providing, with the heterologous sequence, a transcriptional regulatory sequences that are active in eukaryotic cells. Such transcriptional regulatory sequences can include cellular promoters such as, for example, the viral promoters such as, for example, herpesvirus, adenovirus and papovavirus promoters and DNA copies of retroviral long terminal repeat (LTR) sequences. In such embodiments, the heterologous gene is introduced in an expression construct in which the heterologous gene is operatively linked to such transcriptional regulatory elements.

The heterologous (i.e., foreign) nucleotide sequence that is incorporated into the adenovirus can consist of one or more gene(s) of interest or other nucleotide sequences that are not genes but have other functions, and preferably of therapeutic interest. In the context of the present invention, a nucleotide sequence or gene of interest can code either for an antisense RNA, short hairpin RNA, a ribozyme or for an mRNA which will then be translated into a protein of interest. Such a nucleotide sequence or gene may comprise genomic DNA, complementary DNA (cDNA) or of mixed type (minigene, in which at least one intron is deleted). The nucleotide sequence or gene can encode a regulatory or therapeutic function, a mature protein, a precursor of a mature protein, in particular a precursor that comprises a signal peptide, a chimeric protein originating from the fusion of sequences of diverse origins, or a mutant of a natural protein displaying improved or modified biological properties. Such a mutant may be obtained by, deletion, substitution and/or addition of one or more nucleotide(s) of the gene coding for the natural protein, or any other type of change in the sequence encoding the natural protein, such as, for example, transposition or inversion.

The gene that is being delivered by the vector may be placed under the control of elements (DNA control sequences) suitable for its expression in a host cell. Suitable DNA control sequences are understood to mean the set of elements needed for transcription of a gene into RNA (antisense RNA or mRNA) and for the translation of an mRNA into protein. For example, these elements would include at least a promoter. The promoter may be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention may be modified so as to contain regulatory sequences. Exemplary promoters may include tissue specific promoters when the gene is to be targeted to a given tissue type. Other conventional promoters that may be used include but are not limited to the HSV-I TK (herpesvirus type 1 thymidine kinase) gene promoter, the adenoviral MLP (major late promoter), the RSV (Rous Sarcoma Virus) LTR (long terminal repeat), the CMV immediate early promoter, SV-40 immediate early promoter, and the PGK (phosphoglycerate kinase) gene promoter, for example, permitting expression in a large number of cell types.

The genes to be delivered by the adenoviral vectors may be any genes including but not limited to genes that encode cytokines such as interferons and interleukins; genes encoding lymphokines; genes coding for membrane receptors such as the receptors recognized by pathogenic organisms (viruses, bacteria or parasites), preferably by the HIV virus (human immunodeficiency virus); genes coding for coagulation factors such as factor VIII and factor IX; genes coding for dystrophins; genes coding for antigenic epitopes in order to increase the host cell's immunity; genes coding for major histocompatibility complex classes I and II proteins, as well as the genes coding for the proteins which are inducers of these genes; genes coding for antibodies; genes coding for immunotoxins; genes encoding toxins; genes encoding growth factors or growth hormones; genes encoding cell receptors and their ligands; genes encoding tumor suppressors; genes involved in cardiovascular disease including, but not limited to, oncogenes; genes encoding growth factors including, but not limited to, fibroblast growth factor (FGF), vascular endothelial growth factor (VEGF), and nerve growth factor (NGF); e-nos, tumor suppressor genes including, but not limited to, the Rb (retinoblastoma) gene; lipoprotein lipase; superoxide dismutase (SOD); catalase; oxygen and free radical scavengers; apolipoproteins; and pai-1 (plasminogen activator inhibitor-1); genes coding for cellular enzymes or those produced by pathogenic organisms; and suicide genes.

In certain preferred embodiments the vaccines of the present invention are prepared to vaccinate swine against causing diseases in those animals. For example, the vaccines may be directed to pseudorabies virus (PRV) gp50; transmissible gastroenteritis virus (TGEV) S gene; porcine rotavirus VP7 and VP8 genes; genes of porcine respiratory and reproductive syndrome virus (PRRS), in particular ORFs 3, 5 and 7; genes of porcine epidemic diarrhea virus; genes of hog cholera virus; genes of porcine parvovirus; and genes of foot-and-mouth disease virus; genes associated with porcine circovirus; and genes of porcine influenza virus. Representative bovine pathogen antigens include bovine herpes virus type 1; bovine diarrhea virus; bovine coronavirus; and genes of foot-and-mouth disease virus. Representative human pathogen antigens include but are not limited to HIV virus antigens and hepatitis virus antigens.

Cytokines and growth factors such as vascular endothelial growth factor (VEGF), epidermal growth factor, fibroblast growth factor, pleiotrophin, platelet-derived growth factor, erythropoietin, stem-cell factor (SCF), TNF-α; an interferon such as interferon-γ, interferon β, interferon-α, granulocyte-colony-stimulating-factor (G-CSF) granulocyte-macrophage colony stimulating factor (GM-CSF); stromal cell-derived factor-1, macrophage colony stimulating factor, RANTES, IGF-I, SDF-I, MIP Iα, MCP-I and MCP-2, eotaxin, eotaxin3, eotaxin4, LKNI, MPIF-2 and LD78beta, Leukemia Inhibitory Factor (LIF) interleukins such as e.g., IL-I, IL2, IL-3, IL4, IL-5, IL-6, IL-7, IL-8. IL-9, ILIO, IL-II, IL-12, IL-13, IL-14, IL-15, IL-16, IL-17, IL-18, IL-19, IL-20, IL-21, IL-22, IL-23, IL-24, IL-25, TOLL-like receptors and ligands, integrin receptors, and the like also may be delivered using the vectors of the present invention.

It should be understood that while in some circumstances it might be desirable to incorporate the whole gene into the vector, other vectors can be constructed that comprise only a portion of the nucleotide sequences of genes can be used (where these are sufficient to generate a protective immune response or a specific biological effect) rather than the complete sequence as found in the wild-type organism. Where the genes contain a large number of introns, a cDNA may be preferred.

The adenoviral vectors may comprise more than one foreign gene. The methods of the invention can be used to provide protection against a wide variety of diseases affecting pigs, humans, cattle, and other mammals. Any of the recombinant antigenic determinants or recombinant live viruses of the invention can be formulated and used in substantially the same manner as described for antigenic determinant vaccines or live vaccine vectors

In one embodiment, provided herein is a recombinant PAdV-3 vector optimized for increased yield and/or expression and/or encapsidation efficiency in a porcine cell line, wherein the optimized PAdV-3 is a chimeric PAdV-3 comprising no less than 3 TTTG and/or TTTC packaging signal sites operably linked between the 5′ inverted terminal repeat (5′ ITR) and the E1A early transcription region. In another embodiment, provided herein is a porcine cell line expressing the optimized chimeric vectors, viruses or both as described herein. In one embodiment, the porcine cell line transfected with the optimized vectors and/or viruses as described herein is swine testicular cell line (ST). In another embodiment, the porcine cell line transfected with the optimized vectors and/or viruses as described herein, is porcine kidney cell line 15 (PK-15).

Also provided herein is a pharmaceutical composition comprising a chimeric PAdV-3 optimized for increased yield and/or expression and/or encapsidation efficiency, comprising no less than 3 TTTG and/or TTTC packaging signal sites operably linked between the 5′ inverted terminal repeat (5′ ITR) and the E1A early transcription region. In one embodiment, the chimeric PAdV-3 optimized for increased yield and/or expression and/or encapsidation efficiency, comprises a chimeric nucleotide sequence encoding for a non-PAdV-3 adenovirus packaging signaling region, comprising the region between 5′ ITR and E1A of the non-PAdV-3 adenovirus, operably linked to the 5′ side of the early transcription region E1A of the PAdV-3 genome.

The pharmaceutical compositions comprising a therapeutically effective amount of a recombinant adenovirus vector, recombinant adenovirus or recombinant protein, prepared according to the methods of the invention, in combination with a pharmaceutically acceptable vehicle and/or an adjuvant can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially), by oral administration, by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

The invention further provides for methods of treatment in which a therapeutically effective amount of a recombinant PAdV-3 adenoviral vector that has been prepared according to the methods described herein is administered to a mammalian subject requiring treatment.

The antigens used in the present invention can be either native or recombinant antigenic polypeptides or fragments. They can be partial sequences, full-length sequences, or even fusions (e.g., having appropriate leader sequences for the recombinant host, or with an additional antigen sequence for another pathogen). The preferred antigenic polypeptide to be expressed by the virus systems of the present invention contain full-length (or near full-length) sequences encoding antigens. Alternatively, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. Preferably the peptide should encode a “protective epitope” that is capable of raising in the host a “protective immune response;” i.e., an antibody- and/or a cell-mediated immune response that protects an immunized host from infection.

Genes for desired antigens or coding sequences thereof which can be inserted include those of organisms which cause disease in mammals, particularly bovine pathogens such as foot-and-mouth disease virus, bovine rotavirus, bovine coronavirus, bovine herpes virus type 1, bovine respiratory syncytial virus, bovine parainfluenza virus type 3 (BPI-3), bovine diarrhea virus, Pasteurella haemolytica, Haemophilus somnus and the like. Genes encoding antigens of human pathogens also useful in the practice of the invention. The vaccines of the invention carrying foreign genes or fragments can also be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. Oral and/or intranasal vaccination may be preferable to raise mucosal immunity (which plays an important role in protection against pathogens infecting the respiratory and gastrointestinal tracts) in combination with systemic immunity.

The dosage for all routes of administration of in vivo recombinant virus vaccine depends on various factors including, the size of host/patient, nature of infection against which protection is needed, carrier and the like and can readily be determined by those of skill in the art. By way of non-limiting example, a dosage of between 10³ pfu and 10¹⁵ pfu, preferably between 10⁴ and 10¹³ pfu, more preferably between 10⁵ to 10¹¹ pfu and the like can be used.

The invention also includes a method for providing gene delivery to pigs, however, it is possible that the PAdV-3 vectors may be used for gene delivery to another mammal, such as a bovine, human, ovine, canine, feline, or other mammal in need thereof, to control a gene deficiency, to provide a therapeutic gene or nucleotide sequence and/or to induce or correct a gene mutation.

The methods of use of the vectors of the invention can be used, for example, in the treatment of conditions including, but not limited to hereditary disease, infectious disease, cardiovascular disease, and viral infection. These kinds of techniques are currently being used by those of skill in the art for the treatment of a variety of disease conditions. Examples of foreign genes, nucleotide sequences or portions thereof that can be incorporated for use in a conventional gene therapy include, cystic fibrosis transmembrane conductance regulator gene, human minidystrophin gene, alpha-1-antitrypsin gene, genes involved in cardiovascular disease, and the like.

The present invention relates to methods and compositions for increasing the viral titer of recombinantly produced PAdV-3 in cell lines. The efficiency of production of existing PAdV-3 vectors, e.g., PAdV-3 vectors that express the gp55 gene of classical swine fever virus (CSFV) [Hammond et al., 2000] could be greatly increased by modifying the packaging signal of the underlying PAdV-3 to contain three or more TTT^{G}/_{C} signals in between the 5′ITR region and the E1A region. The presence of the extra TTT^{G}/_{C} elements in the packaging signal of the recombinant PAdV-3 increases the encapsidation and titer of the PAdV-3 produced in the cell lines. In so doing, the invention provides the advantage of increased efficiency of production of recombinant PAdV-3 vectors for use in vaccines.

A “host cell” is a cell which has been transformed, or is capable of transformation, by an exogenous DNA sequence. In the present invention, the host cells are those that can support the replication of a PAdV-3 adenoviral vector (i.e., can become infected by the adenovirus and allow the adenovirus to replicate therein). “Transformation” of a cell entails introduction of exogenous DNA into the cell.

### Example 1

### Preparation of Virus Titers

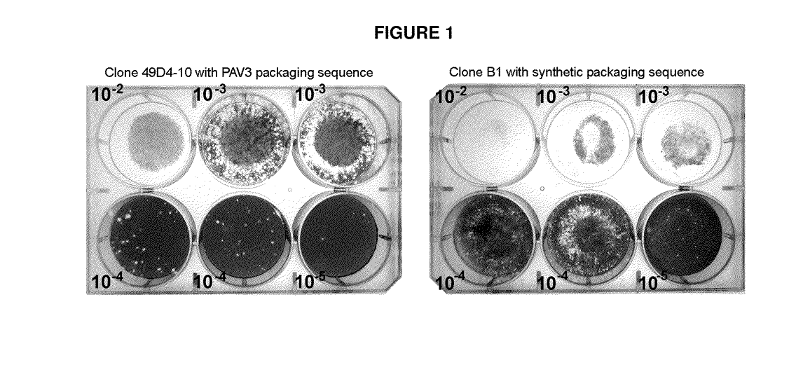

FIG. 1 shows data from virus titers for recombinant PAV3 clones with a wildtype PAV3 or a synthetic packaging sequences expressing CMVp-PRRS ORF6-5/MLP-PRRS ORF7.

Virus titers were determined by the plaque assay method. Monolayers of ST cells were infected using 333 μl of serially diluted samples of virus preparations, then overlayed with agarose. Cells were fed on day 3 and fixed and stained on day 7 before counting plaques. Assay plates were set up in pairs to allow for direct comparisons of the effect of packaging sequences on virus titer. Representative plaque assay plates of virus samples with a PAV3 packaging sequence (Clone 49-D4) and virus with a synthetic packaging sequence (Clone B1) are shown.

| | Virus titers (pfu ml⁻¹) | | Titer difference: |
|---|---|---|---|
| | PAV3 packaging | Synthetic packaging | Synthetic vs |
| Experiment | sequence | sequence | PAV3 |
| 1 | 1.06 × 10⁶ | 1.20 × 10⁷ | 11.3 x |
| 2 | 9.25 × 10⁵ | 1.30 × 10⁷ | 14.1 x |
| 3 | 1.47 × 10⁶ | 1.20 × 10⁷ | 8.2 x |
| 4 | 1.03 × 10⁶ | 1.20 × 10⁷ | 11.7 x |
| Average | 1.12 × 10⁶ | 1.23 × 10⁷ | 10.9 x |

1. A recombinant porcine adenovirus 3 (PAdV-3) vector wherein said vector comprises porcine adenovirus 3 ITR sequences, and at least one heterologous nucleotide sequence, wherein said PAdV-3 genome has a modified packaging signal that comprises at least three TTT^{G}/_{C} packaging elements between the 5′ inverted terminal repeat and the E1A early transcription region.

2. The recombinant PAdV-3 vector of claim 1, wherein said PAdV-3 genome has been modified to include the insertion of two or more TTT^{G}/_{C} packaging elements within the region located between the 5′ inverted terminal repeat and the E1A early transcription region of PAdV-3.

3. The recombinant PAdV-3 vector of claim 1, wherein said PAdV-3 genome has been modified to replace the wild-type packaging signal of PAdV-3 located between the 5′ inverted terminal repeat and the E1A early transcription region of wild-type PAdV-3 with the LHE packaging signal domain from a non-PAdV-3 virus.

4. The recombinant PAdV-3 vector of claim 1, wherein said non-PAdV-3 virus is selected from the group consisting of human adenovirus type 5 (HAdV-5), canine adenovirus type 1 (CAdV-1), porcine adenovirus type 5 (PAdV5) and bovine adenovirus type 3 FAV-9 or artificial sequence.

5. The recombinant PAdV-3 of claim 1, wherein the packaging signal element has the general form:
TTT^{G}/_{C}XₙCG wherein X is a nucleic acid sequence of length “n” and “n” is an integer between 5 and 15.

6. The recombinant PAdV-3 vector of claim 1, wherein said vector is replication competent.

7. The recombinant adenoviral vector of claim 6, wherein said recombinant PAdV-3 comprises a heterologous nucleotide sequence inserted into a nonessential region of the adenoviral genome.

8. The recombinant PAdV-3 vector of claim 6, wherein said vector comprises a deletion in a region selected from the group consisting of the PAdV-3 E2 region, E3 region, E4 region, L1 region, L2 region, L3 region, L4 region, L5 region, L6 region and the region between E4 region and the right end of the genome.

9. The recombinant PAdV-3 vector of claim 1, wherein said vector is replication defective.

10. The recombinant porcine adenoviral vector of claim 9, wherein said recombinant PAdV-3 comprises a heterologous nucleotide sequence inserted into an essential region of the PadV-3.

11. The recombinant PAdV-3 vector of claim 9, wherein said replication-defective vector comprises a defective or deleted E1 function.

12. The recombinant PAdV-3 vector of claim 7, wherein said non-essential region is selected from the group consisting of the E3 region, ORF 1-2 and 4-7 of E4, the region between the end of E4 and the ITR of the porcine adenovirus genome.

13. The method of claim 7, wherein said heterologous nucleotide sequence is a gene that encodes a protein selected from the group consisting of an immunomodulator, an antigen, a pathogen, an immunogenic polypeptide, a therapeutic polypeptide, a growth hormone, and a cytokine.

14. An improved recombinant PAdV-3 vector, comprising ITR sequences, packaging sequences, at least heterologous nucleotide sequence, said improvement comprising an optimized PAdV-3 genome having at least 3 TTTG and/or TTTC packaging signal elements between the 5′ inverted terminal repeat and the E1A early transcription region of said PAdV-3 genome.

15. A host cell comprising an adenovirus vector of claim 1, wherein said host cell is capable of being infected by porcine adenovirus 3, and has an increased titer of PAdV-3 production when cultured under conditions which permit packaging of said chimeric PAdV-3 into a viral capsid as compared to a host cell infected by PAdV-3 having a wild-type packaging signal that contains two or fewer TTT^{G}/_{C} elements.

16. The host cell according to claim 15, wherein said cell is a mammalian cell.

17. The host cell according to claim 16, wherein said mammalian cell is a recombinant porcine cell.

18. A composition capable of inducing an immune response in a mammalian subject, said composition comprising a recombinant adenoviral vector of claim 1 and a pharmaceutically acceptable excipient.

19. A method for eliciting an immune response in a mammalian subject comprising administering a composition of claim 18 to the mammalian subject.

20. The method of claim 19, wherein said mammalian subject is a pig.

21. A method of increasing the titer of production of a recombinant porcine adenovirus in a host cell capable of being infected by porcine adenovirus 3 comprising introducing into said porcine host cells an optimized recombinant PAdV-3 of claim 1, wherein presence of said optimized packaging signal in said PAdV-3 increases the titer of said recombinant PAdV-3 from said a porcine host cell culture as compared to a recombinant PAdV-3 vector prepared from wild-type PAdV-3 that contains only one or two TTTG/C elements between the 5′ITR and the E1A region of the PAdv-3 genome.

22. The method of claim 21, wherein chimeric nucleotide sequence encoding for a non-PAdV-3 adenovirus packaging signaling region is the LHE packaging signal domain of human adenovirus type 5 (HAdV-5), comprising nucleotides 194 to 358.

23. The method of claim 21, wherein the chimeric nucleotide sequence encoding for a non-PAdV-3 adenovirus packaging signaling region comprises a plurality of TTTG and/or TTTC elements.

24. The method of claim 21, wherein chimeric nucleotide sequence encoding for a non-PAdV-3 adenovirus packaging signaling region comprises no less than 3 TTTG and/or TTTC elements.

25. The method of claim 21, wherein the packaging signal region is any one of the nucleotide sequence represented by SEQ. ID. NOs. 1, 2, 3, 4, 6 or 7.

26. The method of claim 21, wherein the step of growing said porcine host cells in culture comprises culturing said porcine host cell under conditions which permit packaging of said chimeric PAdV-3 into a viral capsid.

27. The method of claim 21, wherein the porcine host cell is a swine testicle cell line (TS), a porcine kidney cell line (PK-15).

28. The method of claim 21, wherein presence of said optimized packaging signal produces at least a two log-fold increase in titer of said recombinant PAdV-3 in porcine cell lines as compared to growth of recombinant PAdV-3 prepared with a wild-type packaging signal.

29. The method of claim 21, wherein the titer of said PAdV-3 is increased to between 10⁶ pfu/ml to about 10¹² pfu/ml.

30. A pharmaceutical composition comprising the optimized porcine adenovirus type 3 (PAdV-3) according to claim 1 and a physiologically compatible carrier.


SEQUENCE LISTING (7 sequences)

SEQ ID NO: 1 - DNA, 460 bp, Artificial Sequence
  LHE of human adenovirus serotype 5 (HAdV-5)
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatag

SEQ ID NO: 2 - DNA, 400 bp, Artificial Sequence
  LHE of canine adenovirus type 1 (CAdV-1)
catcatcaataatatacaggacaaagaggtgtggcctaaatgttgtttttttttttaaaaagtttttgtttgattgttttgacaaggttacaccctgttcagggcgtttcccacgggaaagaccatgacgtcaattgggtgtttttgtggactttggcccgcgagcccgtttttgactgtttactgaaattgtggtttttgggtggagtgtttactcatgtcagaggaaattttctgggtttattgttctagccttggagccaattttccacacccggcgccagactgtctgacattgttttgagaaactctccgttcccagggtcacaggaccgcacccgcgagaatggtcacgccttttgttttttctgctttaaattcactgcagagacgaaagagg

SEQ ID NO: 3 - DNA, 500 bp, Artificial Sequence
  LHE of bovine adenovirus type 3 (BAdV-3)
catcatcaataatctacagtacactgatggcagcggtccaactgccaatcatttttgccacgtcatttatgacgcaacgacggcgagcgtggcgtgctgacgtaactgtggggcggaggcgtcgcggaggcggcggcgctgggcggggctgagggcggcgggggcggcgcgcggggcggcgcgcggggcggggcgaggggcggagttccgcacccgctacgtcattttcagacattttttagcaaatttgcgccttttgcaagcatttttctcacatttcaggtatttagagggcggatttttggtgttcgtacttccgtgtcacatagttcactgtcaatcttcattacggcttagacaaattttcggcgtcttttccgggtttatgtccccggtcacctttatgactgtgtgaaacacacctgcccattgtttacccttggtcagttttttcgtctcctagggtgggaacatcaagaacaaatttgccgagtaattgt

SEQ ID NO: 4 - DNA, 330 bp, Artificial Sequence
  LHE of porcine adenovirus type 5 (PAdV-5)
cccatgggtgaggtctggacatgcccagacacgtcagagctatttgacaaaggtcactttcgaccgcgcctgtggaatttcccccgtacagcccattttcgtcatggactccgcctatttcacactataagagcaggcgacacagttctctccacattcgattcgagactcgccgtacacggacacaccacaaagtaagttgaaatgagttcagacaggcttctttttgtgaggctttgtagggacgctgttcctcctcgccgggcatcgccaggcgctgctggttatgatttatgttctacaaactacgtagtcgtgagggcgcgggat

SEQ ID NO: 5 - DNA, 460 bp, Artificial Sequence
  LHE of PAdV-3
catcatcaataatataccgcacacttttattgccccttttgtggcgtggtgattggcggagagggttgggggcggcgggcggtgattggtggagaggggtgtgacgtagcgtgggaacgtgacgtcgcgtgggaaaatgacgtgtgatgacgtcccgtgggaacgggtcaaagtccaaggggaaggggtggagccctggggcggtcctccgcggggcggggccgagcggcggaaattcccgcacaggtggagagtaccgcgggattttgtgccctctggaccggaccttcgccctccggtgtggcacttccgcaccacacgtccgcggcccggtattccccacctgacgacggtgacaccactcacctgagcggggtgtccttcgcgctgagaggtccgcggcggccgcccgagatgacgtgtgtgggtgtattttttcccctcagtgtatatagtccgc

SEQ ID NO: 6 - DNA, 459 bp, Artificial Sequence
  Chimeric LHE for use in recombinant PAdV-3
catcatcaataatataccgcacacttttattgccccttttgtggcgtggtgattggcggagagggttgggggcggcgggcggtgattggtggagaggggtgtgacgtagcgtgggaacgtgacgtcgcgtgggaaaatgacgtgtgatgacgtcccgtgggaacgggtcaaagtccaaggggaaggggtggagccctggggcggtcctccgcggggcggggccgagcggcggaaattcccgcacaggtggagagtaccgcgggattttgtgccctctggaccggaccttcgccctttggtgtggcacttccgcatttgacgtccgcggcccggttttgcccacctgacgacggtgtttgcactcacctgagcggggtgtcctttgcgctgagaggtccgcggtttcgcccgagatgacgtgtgtgggtgtattttttcccctcagtgtatatagtccgc

SEQ ID NO: 7 - DNA, 849 bp, Artificial Sequence
  FAV-9
catcatctatatatacctacatgaatgacgctgctgtcacctcgcactaccgctcccgcccgctagatggcaaaattgctccaccaaagtatttccgtgatacaaagtccggactgggcgcggcgaatacgtgccgggcgaggcacggagccgactttctggcaccgatagcaataaatgggcgttcccttgcgtgcggaattgcgccggcacagtcccaatggcttgggagcggtccagaaagcgattagtttttgcgtacgcacgcgaaagctatttttgcagcacttacttccttatttgtttacaaccgcctccgcctgtgtgtaatttaaaatttgtacataccgttccgatcgccgtacttgggcttggaacgcggacttcgagtccctaaattgatttatggtgtcttaatcagcgctttttgtgccacgttgtgcgttctccgttgacctttgaaaaggcgcgtggtatttctcggtgtgtttttcgggcggattacgcgttacgtgatcgttttatggcgggctccgggcgtataacagtggagcggctgatcgtcagagcacggatgagcgagcagccggactcaccgccggagctctatccgtctctcctggtcctgcgggtgaacccggttccccgtactccgttcgttttggacatcagccgtttgcccgtggtgccggagcgcattttgctgcacgactttccgaacggaccctggtcggaaacgatttggtacgggaagatcactcagcgggagctgaaccaggcgctagagtctatcgtggacgtgctttaggtaatttcctccgttctttttcccgtttaggtgaggagatg